(19) United States
(12) Patent Application Publication
(10) Pub. No.: US 2023/0276809 A1
(43) Pub. Date: Sep. 7, 2023

(54) USE OF PENICILLIUM EHRLICHII IN CONTROLLING PLANT PESTS, AND A CONTROL METHOD

(71) Applicant: Anhui Agricultural University, Hefei City (CN)

(72) Inventors: Hao JIANG, Hefei City (CN); Hui WU, Hefei City (CN); Jiaming LIU, Hefei City (CN); Mengting ZHANG, Hefei City (CN); Yanqiu LI, Hefei City (CN); Li QIN, Hefei City (CN); Daxiang LI, Hefei City (CN); Chuankui SONG, Hefei City (CN); Xiaochun WAN, Hefei City (CN)

(21) Appl. No.: 17/744,222

(22) Filed: May 13, 2022

(30) Foreign Application Priority Data
Mar. 2, 2022 (CN) 202210196770.2

(51) Int. Cl. A01N 63/36; C12N 1/14

(52) U.S. Cl. CPC A01N 63/36; C12N 1/145; C12R 2001/80

(57) ABSTRACT

The present disclosure relates to the technical field of plant pests control, in particular to use of Penicillium ehrlichii Z19 in controlling plant pests, and a control method. The Penicillium ehrlichii Z19 has a deposit number of CCTCC NO: M 2021635. The Penicillium ehrlichii Z19 has a desirable insecticidal activity against lepidopteran pests, especially has a 7-day insecticidal effect of not less than 80% on Helicoverpa armigera, Plutella xylostella, Ectropis obliqua, and Spodoptera exigua. In addition, compared with traditional chemical control methods, the method for controlling pests using the Penicillium ehrlichii Z19 in the present disclosure can control the plant pests in a safe, efficient and pollution-free manner. Therefore, the method reduces pollutions to ecological environment, improves quality and safety of agricultural products, and protects human health.

### TECHNICAL FIELD

The present disclosure relates to the technical field of plant pests control, in particular to use of Penicillium ehrlichii Z19 in controlling plant pests, and a control method.

### BACKGROUND ART

Plant endophytes refer to microbial groups that can live in intercellular spaces or cells of various plant organs and tissues, are temporarily non-pathogenic to plants, and have established a harmonious symbiotic relationship with plants.

Plant endophytes colonize host plants, and can promote plant growth; some endophytes can also enhance a resistance of host plants to biotic (such as plant pathogens, pests, etc.) and abiotic stresses. Plant endophytes themselves produce or promote host plants to produce a series of secondary metabolites with a high biological activity, thus exhibiting relatively desirable insecticidal or bacteriostatic activities. Endophytes are now considered as natural resources for biological control, and are widely used in green control of plant diseases and pests due to their safety and high activity. Therefore, endophytes have broad use potentials and development values.

Tea plants (Camellia sinensis) as a perennial woody plant are an important economic crop in China. At present, there are relatively few studies on endophytes of tea plant, and there are even no relevant reports on use of endophytes of tea plant in controlling main lepidopteran pests of agricultural and forestry plants. Therefore, developing use of endophytes of tea plant as microbial pesticides instead of chemical pesticides to control pests severely impairing growth of agricultural and forestry plants, is of great significance to quality safety, green, healthy and sustainable development of agriculture and agricultural products.

### SUMMARY

To solve the above problems, the present disclosure provides use of Penicillium ehrlichii Z19 in controlling plant pests, and a control method. The Penicillium ehrlichii Z19 has a desirable insecticidal activity against lepidopteran pests, especially has a 7-day insecticidal effect of not less than 80% on Helicoverpa armigera. Plutella xylostella, Ectropis obliqua, and Spodoptera exigua.

To achieve the above objective, the present disclosure provides following technical solutions.

The present disclosure provides use of Penicillium ehrlichii Z19 in controlling plant pests, where the Penicillium ehrlichii Z19 has a deposit number of CCTCC NO: M 2021635.

Preferably, the plant pests include lepidopteran pests.

Preferably, the plant pests include one or more of Ectropis obliqua, Spodoptera exigua, Spodoptera frugiperda, Spodoptera litura, Plutella xylostella, Pyrausta nubilalis, Mythimna separata, and Helicoverpa armigera.

The present disclosure further provides use of Penicillium ehrlichii Z19 in preparation of a formulation for controlling plant pests, where the Penicillium ehrlichii Z19 has a deposit number of CCTCC NO: M 2021635.

Preferably, the plant pests include lepidopteran pests.

The present disclosure further provides a method for controlling plant pests, including following steps:

applying a microbial agent comprising Penicillium ehrlichii Z19 on a plant; wherein the Penicillium ehrlichii Z19 has a deposit number of CCTCC NO: M 2021635.

Preferably, the microbial agent has not less than 2×10⁶ of Penicillium ehrlichii Z19 spores per milliliter or per gram of the microbial agent.

Preferably, a medium for preparing the microbial agent includes a potato dextrose agar (PDA) medium.

Preferably, the plant pests include lepidopteran pests.

Preferably, the plant pests include one or more of Ectropis obliqua, Spodoptera exigua, Spodoptera frugiperda, Spodoptera litura, Plutella xylostella, Pyrausta nubilalis. Mythimna separata, and Helicoverpa armigera.

The present disclosure has following beneficial effects:

The present disclosure provides use of Penicillium ehrlichii Z19 in controlling plant pests, wherein the Penicillium ehrlichii Z19 has a deposit number of CCTCC NO: M 2021635. The Penicillium ehrlichii Z19 has a desirable insecticidal activity against lepidopteran pests, especially has a 7-day insecticidal effect of not less than 80% on Helicoverpa armigera, Plutella xylostella, Ectropis obliqua, and Spodoptera exigua.

In addition, compared with traditional chemical control methods, method for controlling pests using the Penicillium ehrlichii Z19 can control the plant pests in a safe, efficient and pollution-free manner. Therefore, the method reduces pollutions to ecological environment, improves quality and safety of agricultural products, and protects human health.

### Description of Biological Deposit

The Penicillium ehrlichii Z19 was deposited in China Center For Type Culture Collection (CCTCC) in Wuhan University at No. 299 Bayi Road. Wuchang District, Wuhan City, Hubei Province on May 31, 2021. with a deposit number of CCTCC NO: M 2021635.

### BRIEF DESCRIPTION OF THE DRAWINGS

To explain technical solutions in examples of the present disclosure or in prior art more clearly, drawings required in examples are described below in brief.

FIG. 1 shows an insecticidal activity effect of Penicillium ehrlichii Z19 on Helicoverpa armigera for 1 d to 7 d;

FIG. 2 shows an insecticidal activity effect of Penicillium ehrlichii Z19 on Plutella xylostella for 1 d to 7 d;

FIG. 3 shows an insecticidal activity effect of Penicillium ehrlichii Z19 on Ectropis obliqua for 1 d to 7 d;

FIG. 4 shows an insecticidal activity effect of Penicillium ehrlichii Z19 on Spodoptera exigua for 1 d to 7 d;

FIG. 5 shows an insecticidal activity effect of Penicillium ehrlichii Z19 on Spodoptera litura for 1 d to 7 d;

FIG. 6 shows an insecticidal activity effect of Penicillium ehrlichii Z19 on Mythimnaseparata for 1 d to 7 d;

FIG. 7 shows an insecticidal activity effect of Penicillium ehrlichii Z19 on Pyrausta nubilalis for 1 d to 7 d; and

FIG. 8 shows an insecticidal activity effect of Penicillium ehrlichii Z19 on Spodoptera frugiperda for 1 d to 7 d.

### DETAILED DESCRIPTION OF THE EMBODIMENTS

The present disclosure provides use of Penicillium ehrlichii Z19 in controlling plant pests, wherein the Penicillium ehrlichii Z19 has a deposit number of CCTCC NO: M 2021635.

In the present disclosure, the plant pests include preferably lepidopteran pests, more preferably one or more of Ectropis obliqua, Spodoptera exigua, Spodoptera frugiperda, Spodoptera litura, Plutella xylostella, Pyrausta nubilalis, Mythimna separata, and Helicoverpa armigera, and further more preferably one or more of the Ectropis oblique, the Spodoptera exigua, the Plutella xylostella, and the Helicoverpa armigera. In the present disclosure, the plant includes preferably a tea plant (Camellia sinensis). The Penicillium ehrlichii Z19 has a desirable insecticidal activity against lepidopteran pests, especially has a 7-day insecticidal effect of not less than 80% on Helicoverpa armigera, Plutella xylostella, Ectropis obliqua, and Spodoptera exigua.

The present disclosure further provides use of Penicillium ehrlichii Z19 in preparation of a formulation for controlling plant pests, wherein the Penicillium ehrlichii Z19 has a deposit number of CCTCC NO: M 2021635.

In the present disclosure, the plant pests include preferably lepidopteran pests, more preferably one or more of Ectropis obliqua, Spodoptera exigua, Spodoptera frugiperda, Spodoptera litura, Plutella xylostella, Pyrausta nubilalis, Mythimna separata, and Helicoverpa armigera, and further more preferably one or more of the Ectropis obliqua, the Spodoptera exigua, the Plutella xylostella, and the Helicoverpa armigera.

The present disclosure further provides a method for controlling plant pests, including following steps:

applying a microbial agent comprising Penicillium ehrlichii Z19 on a plant; wherein the Penicillium ehrlichii Z19 has a deposit number of CCTCC NO: M 2021635.

In the present disclosure, the microbial agent has preferably not less than 2×10⁶ of Penicillium ehrlichii Z19 spores per milliliter or per gram of the microbial agent.

In the present disclosure, a medium for preparing the microbial agent includes preferably a PDA medium; and the microbial agent is prepared at preferably 25° C.

In the present disclosure, plant pests include preferably lepidopteran pests, more preferably one or more of Ectropis obliqua, Spodoptera exigua, Spodoptera frugiperda, Spodoptera litura, Plutella xylostella, Pyrausta nubilalis, Mythimna separata, and Helicoverpa armigera, and further more preferably one or more of the Ectropis obliqua, the Spodoptera exigua, the Plutella xylostella, and the Helicoverpa armigera. In the present disclosure, method for controlling pests using the Penicillium ehrlichii Z19 can control the plant pests in a safe, efficient and pollution-free manner. Therefore, the method reduces pollutions to ecological environment, improves quality and safety of agricultural products, and protects human health.

In order to further describe the present disclosure, use of Penicillium ehrlichii Z19 in controlling plant pests, and control method provided by the present disclosure is described in detail below with reference to examples, but the examples should not be understood as a limitaion of claimed scope herein.

### Example 1

### Isolation and Identification of an Endophytic Strain

Root, stem and leaf tissues of a healthy tea plant were taken, and surface soil was removed with tap water; the tissues were soaked in 70% (volume concentration) alcohol for 1 min, in a 3 wt.% sodium hypochlorite solution for 4 min, and then in 70% (volume concentration) alcohol for 1 s; the tissues were rinsed 5 times with sterile water, and 100 µL of a last rinsed sterile water was imbibed and spread on a PDA medium to prepare a control medium.

Tissue separation method: surface-sterilized tea plant tissues were cut into small pieces of 5 mm * 5 mm, wherein an incision side was sticked on a PDA medium; culture was conducted at 25° C. for 7 d. the tissues were observed every day, and newly-grown mycelium were selected with a bacterial transfer ring for separate culture.

Internal transcribed spacer (ITS) and 18S loci sequencing were conducted on isolated endophytes, and sequencing results were compared on the National Center of Biotechnology Information (NCBI) by Basic Local Alignment Search Tool (BLAST), and a similar strain was named Penicillium ehrlichii (Table 1).

TABLE 1

Identification results of ITS sequencing

| | | | | | | |
|---|---|---|---|---|---|---|
| Accession | Kingdom | Phylum | Class | Order | Family | Reference species |
| NR 121229.1 | Fungi | Ascomycota | Eurotiales | Aspergillaceae | Penicillium | Penicillium ehrlichii |

The screened strain was named Penicillium ehrlichii Z19, and was sent to CCTCC for deposit, with a deposit number of CCTCC NO: M 2021635.

### Example 2

A toxicity test was conducted on 8 plant pests (Ectropis obliqua, Spodoptera exigua, Spodoptera frugiperda, Spodoptera litura, Plutella xylostella, Pyrausta nubilalis. Mythimna separata, and Helicoverpa armigera) using the Penicillium ehrlichii Z19 screened in Example 1, number of dead pests was counted at different treatment times, and a corrected mortality was calculated. Detailed content was as follows:

A strain of Penicillium ehrlichii Z19 was cultured in a PDA medium (at a constant temperature of 25° C.), and conidia of the Penicillium ehrlichii Z19 cultured for 7 d on a PDA plate medium were washed down from the PDA plate with a 0.1‰ Tween-80 solution. Observation was conducted by a hemocytometer, and a spore suspension of 2 × 10⁶ (cells/ml) was prepared with 0.1‰ Tween-80 solution.

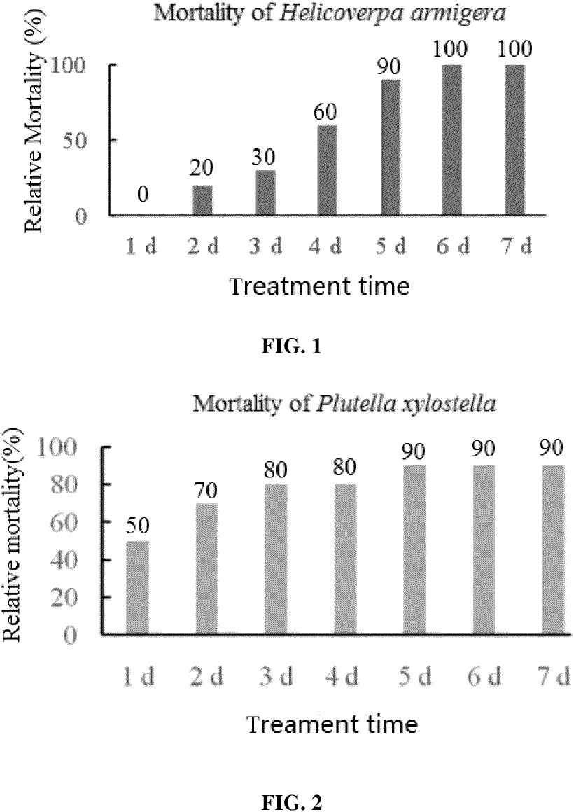

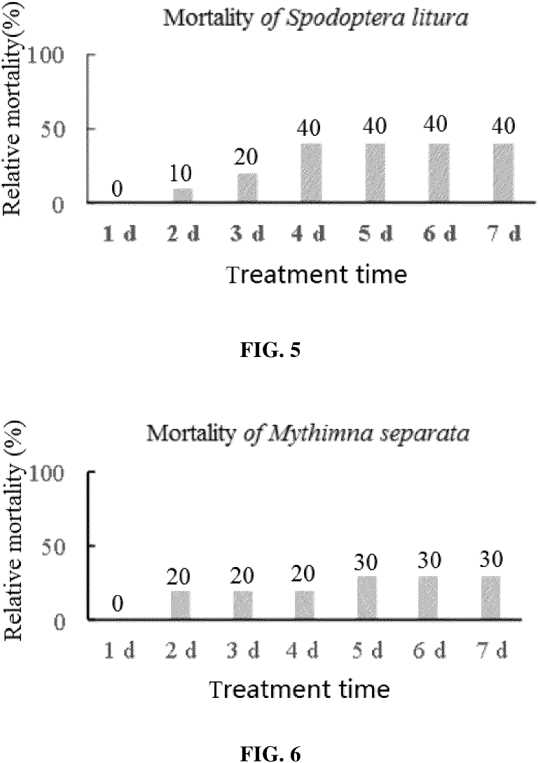

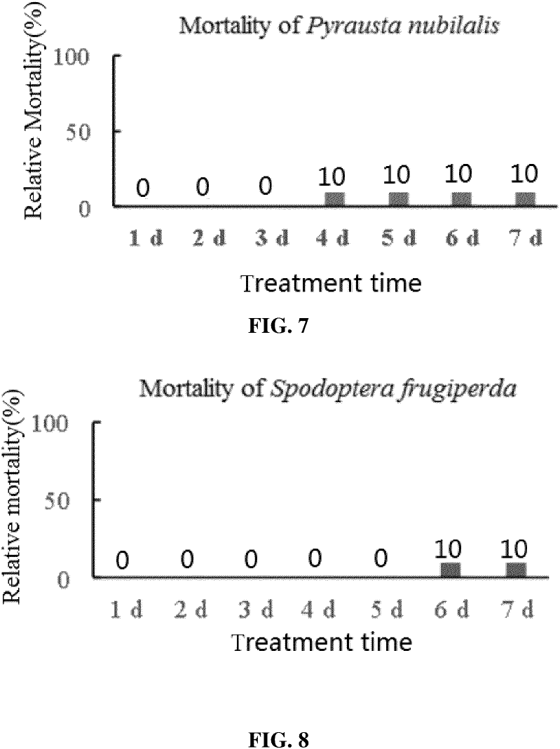

The toxicity test of tested pests was conducted by a spore suspension immersion method. A treatment groups were as follows: 2-instar larvae of the tested pests were infested with the spore suspension for 2 s, respectively, followed by taking out, and conducting raising separately, wherein 30 pests of each treatment were tested; health status was observed every day and number of dead pests was counted; meanwhile, a control group was set up for each treatment group (treatments were similar to the treatment group, a only difference was that the spore suspension was replaced with a 0.1‰ Tween-80 solution; and a corrected mortality was calculated. A test results are shown in FIG. 1 to FIG. 8. A formula for calculating the corrected mortality was as follows:

$$\begin{matrix}\text{Corrected mortality} \left(\%\right) = \\ \left(\text{mortality of treatment group - mortality of control}\right) \\ \left(\text{group}\right) / \left(1 \text{- mortality of control group}\right) \times 100.\end{matrix}$$

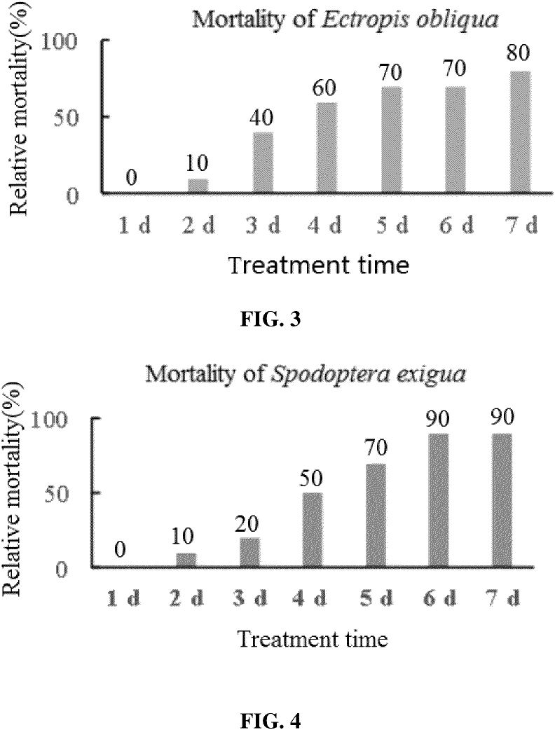

From FIG. 1 to FIG. 8, it could be seen that the Penicillium ehrlichii Z19 had a certain insecticidal activity against selected 8 plant pests; in particular, an insecticidal toxicity against Helicoverpa armigera reached 60% on 4 d, 90% on 5 d, and 100% on 6 d (FIG. 1); an insecticidal toxicity against Plutella xylostella reached 50% on 1 d, 70% on 2 d. 80% on 3 d, 90% on 5 d, and maintains 90% at 6 d and 7 d (FIG. 2); an insecticidal toxicity against Ectropis obliqua reached 60% on 4 d, 70% on 5 d, and 80% on 7 d (FIG. 3); and an insecticidal toxicity against Spodoptera exigua reached 50% on 4 d, 70% on 5 d, and 90% on 6 d (FIG. 4).

In summary, the Penicillium ehrlichii Z19 has a desirable insecticidal activity against lepidopteran pests, especially has a 7-day insecticidal effect of not less than 80% on Helicoverpa armigera, Plutella xylostella, Ectropis obliqua, and Spodoptera exigua.

The present disclosure has been disclosed with preferred examples as above, which shall not be construed as a limitation to the present disclosure. Any person skilled in the art can make changes and modifications without departing from the spirit and scope of the present disclosure. The present disclosure shall fall within the claimed scope defined in the claims.

What is claimed is:

1. Use of Penicillium ehrlichii Z19 in controlling plant pests, wherein the Penicillium ehrlichii Z19 has a deposit number of CCTCC NO: M 2021635.

2. The use according to claim 1, wherein the plant pests comprise lepidopteran pests.

3. The use according to claim 1, wherein the plant pests comprise one or more of Ectropis obliqua, Spodoptera exigua, Spodoptera frugiperda, Spodoptera litura, Plutella xylostella, Pyrausta nubilalis, Mythimna separata, and Helicoverpa armigera.

4. Use of Penicillium ehrlichii Z19 in preparation of a formulation for controlling plant pests, wherein the Penicillium ehrlichii Z19 has a deposit number of CCTCC NO: M 2021635.

5. The use according to claim 4, wherein the plant pests comprise lepidopteran pests.

6. A method for controlling plant pests, comprising following steps: applying a microbial agent comprising Penicillium ehrlichii Z19 on a plant; wherein the Penicillium ehrlichii Z19 has a deposit number of CCTCC NO: M 2021635.

7. The method according to claim 6, wherein the microbial agent has not less than 2×10⁶ of Penicillium ehrlichii Z19 spores per milliliter or per gram of the microbial inoculum.

8. The method according to claim 6, wherein a medium for preparing the microbial agent comprises a PDA medium.

9. The method according to claim 6, wherein the plant pests comprise lepidopteran pests.

10. The method according to claim 6, wherein the plant pests comprise one or more of Ectropis obliqua, Spodoptera exigua, Spodoptera frugiperda, Spodoptera litura, Plutella xylostella, Pyrausta nubilalis, Mythimna separata, and Helicoverpa armigera.

11. The use according to claim 3, wherein the plant pests comprise lepidopteran pests.

12. The method according to claim 8, wherein the microbial agent has not less than 2×10⁶ of Penicillium ehrlichii Z19 spores per milliliter or per gram of the microbial inoculum.

13. The method according to claim 9, wherein the microbial agent has not less than 2×10⁶ of Penicillium ehrlichii Z19 spores per milliliter or per gram of the microbial inoculum.

14. The method according to claim 10, wherein the microbial agent has not less than 2×10⁶ of Penicillium ehrlichii Z19 spores per milliliter or per gram of the microbial inoculum.
